(19) United States
(12) Patent Application Publication
(10) Pub. No.: US 2021/0338711 A1
(43) Pub. Date: Nov. 4, 2021

(54) AQUEOUS ANTI MICROBIAL COMPOSITION USEFUL AS A THERAPEUTIC MATERIAL

(71) Applicant: Tygrus, LLC, Troy, MI (US)

(72) Inventors: Lawrence Carlson, Oxford, MI (US); Anthony Atala, Winston Salem, NC (US); Patrick A. Scalera, Canton, MI (US); Andrew M. Yaksic, Brighton, MI (US)

(21) Appl. No.: 17/246,887

(22) Filed: May 3, 2021

Related U.S. Application Data
(60) Provisional application No. 63/144,305, filed on Feb. 1, 2021.
(60) Provisional application No. 63/019,258, filed on May 1, 2020.
(60) Provisional application No. 63/121,856, filed on Dec. 4, 2020.

(51) Int. Cl. A61K 33/00; A61K 47/02; A61K 47/12; A61K 9/00; A61P 31/14

(52) U.S. Cl. CPC A61K 33/00; A61K 47/02; A61P 31/14; A61K 9/0078; A61K 47/12

(57) ABSTRACT

A therapeutic material active against SARS-COV 2 and other microbial pathogens and method of administering the same.

### BACKGROUND

The present application is a non-provisional utility patent application that claims the benefit of priority to U.S. Provisional Application Ser. No. 63/019,258, filed May 1, 2020, currently pending, U.S. Provisional Application Ser. No. 63/144,305, filed Feb. 1, 2021, currently pending, and U.S. Provisional Application Ser. No. 63,121,856, filed Dec. 4, 2020, currently pending the contents of both are incorporated by reference herein in their entirety.

The present disclosure is related to materials that are active against one or more microorganisms. More particularly, the present disclosure is directed to aqueous materials that are active against microorganisms such as bacteria, viruses or fungal microbes. Such microorganism can include, but are not limited to, viruses such as SARS-CoV-2.

The need to identify and employ antimicrobial compounds for use in reducing or eliminating infections microorganisms cannot be underestimated. Various types of such microorganisms can cause infections diseases that are a challenge to the health, safety and well-being of people of all ages. Various viral and/or bacterial and/or fungal pathogens can spread readily through populations, infecting many. This is particularly challenging when large numbers of individuals in the affected population lack natural or acquired immunities to the given pathogen.

The need for effective therapeutic materials and treatment methods cannot be overstated, particularly those which can address and slow or reduce the pathogenic load in an individual in order to augment or effect recovery. Similarly, cleaning and sanitizing of surfaces, including, but not limited to, hard surfaces can be a significant contributor to slowing the spread of infectious diseases. Thus, the need for compositions and materials which can be active against pathogens found on surfaces is also important.

For example, in certain situations, the pathogen can be SARS-CoV-2. Coronavirus disease 2019 (COVID-19), due to infection with severe acute respiratory coronavirus 2[SARS-CoV-2; also known as the 2019 novel coronavirus (2019-nCoV)], emerged in Wuhan, China in December 2019, and has resulted in a roughly 3% mortality rate. While this mortality rate is much higher than for influenza (roughly 0.05%), influenza-related hospitalizations and deaths in the US nevertheless are roughly 280,000 and 16,000, respectively, as a result of the much higher prevalence. COVID-19 has spread to all six. Heretofore, there is no known approved treatments or preventions for COVID-19. And for SARS-CoV-2 virus infections, effective post-infection treatments to minimize both short- and long-term clinical consequences are urgently needed.

It would be desirable to provide a formulation or formulations that can active against one or more pathogens in situ in a patient in order to reduce or eliminate one or more respiratory infection symptoms and/or pathogens causing an infection. It is also desirable to provide a method for treating a patient presenting with an infection caused by one or more pathogens or testing positive for pathogens, as would present in or on tissue present in the respiratory tract. It would also be desirable to provide a therapeutic composition and method that can support tissue regeneration and healing in certain situations.

### SUMMARY

Disclosed herein is a composition that exhibits antimicrobial activity against at least one pathogen selected from the group that includes bacteria, viruses, fungi or mixtures thereof in which the composition includes a compound having the chemical formula:

$$⌊ {H}_{x} {O}_{\frac{( x - 1 )}{2}} ⌋ {Z}_{y}$$

wherein x is an odd integer≥3;
y is an integer between 1 and 20; and
Z is one of a monoatomic ion from Groups 14 and 17 having a charge value between =1 and −3 or a polyatomic ion having a charge between −1 and −3.

Also disclosed herein is a therapeutic material composition and procedure that can be efficacious in treating upper and/or lower respiratory conditions precipitated by viral and/or bacterial infections. More, particularly, the therapeutic material composition and procedure disclosed can be employed upon the presentation of acute symptoms assocaited with infection by one or more viruses and/or bacteria. Also disclosed herein is a composition that comprises one or more inorganic molecules that provide the assocaited therapeutic composition with a pH less than 7. In certain embodiments, the composition can include at least one inorganic acid material. In certain embodiments, the composition can include at least one buffering ion such as calcium.

Also disclosed is a therapeutic material comprising a product produced by the process that includes the steps of contacting a volume of a concentrated inorganic acid in liquid form having a molarity of at least 7, a density between 22° and 70° baume and a specific gravity between 1.18 and 1.93 in a reaction vessel with an inorganic hydroxide present in a volume sufficient to produce a solid material present in the resulting composition as at least one of a precipitate, a suspended solid, a colloidal suspension; and removing the solid material from the resulting liquid material, wherein the resulting material is a viscous material having a molarity of 200 to 150 M. The therapeutic material also includes water. The therapeutic material can have a pH less than 7, in certain embodiments less than 5, and in certain embodiments, less than 3.

In certain embodiments, the therapeutic composition can include at least one compound having the general formula:

$$[ {H}_{x} {O}_{\frac{( x - 1 )}{2}} + {( {H}_{2} O )}_{y} ] Z$$

wherein x is an odd integer ≥3;
y is an integer between 1 and 20; and
Z is a polyatomic ion or monoatomic ion.
   Where desired or required, the component Z is one of a monoatomic ion from Groups 14 through 17 having a charge value between −1 and −3 or a polyatomic ion having a charge between −1 and −3 and x is an integer between 3 and 11 and y is an integer between 1 and 10.

Also disclosed is method for treating a microbiological pathogen that includes the step of introducing a composition as disclosed herein into contact with epithelial tissue for a contact interval, wherein the contact results in a reduction of at least one microbiological pathogen assocaited with the human epithelial tissue. In certain embodiments, the composition can be dispensed as vaporized or atomized fluid through a suitable engineered device. In certain embodiments, a composition as disclosed herein can be administered by inhalation an amount of the compositions as disclosed present in a vaporized carrier material over a suitable administration interval. In certain embodiments the therapeutic material present in the composition can be present in the suitable carrier at a concentration greater than 0.1% by volume. Administration of one or more doses of the composition over a defined dose interval can result in reduction of viral titer in the respiratory system of the patient. Viruses that can respond to such treatment include SARS-CoV-2. Administration of one or more doses of the composition as disclosed herein over a defined dose interval may result in accelerated reversal of viral induced damage of respiratory tissue.

Also disclosed is the composition of water and at least one compound produced by the process comprising the steps of contacting a volume of a concentrated inorganic acid in liquid form having a molarity of at least 7, a density between 22° and 70° baume and a specific gravity between 1.18 and 1.93 in a reaction vessel with an inorganic hydroxide present in a volume sufficient to produce a solid material present in the resulting composition as at least one of a precipitate, a suspended solid, a colloidal suspension and removing the solid material from the resulting liquid material, wherein the resulting material is a viscous material having a molarity of 200 to 150 M the composition is used as a therapeutic treatment for bacterial, viral of fungal infection, particularly one presenting in whole or in part in respiratory regions.

### BRIEF DESCRIPTION OF THE DRAWINGS

The various features, advantages and other uses of the present apparatus will become more apparent by referring to the following detailed description and drawing in which:

FIG. 1 mass spectra collected in the positive ionization mode for Dilute Sulfuric Acid w/400 ppm CaSO4 (A), Dilute Sulfuric Acid (B), an embodiment as disclosed herein prepared according to the process outlined in Example I (C), and Reverse Osmosis Water (D);

FIG. 2 are mass spectra collected in the negative ionization mode for Dilute Sulfuric Acid w/400 ppm CaSO4 (A), Dilute Sulfuric Acid (B), and embodiment as disclosed herein prepared according to the process outlined in Example I (C), and Reverse Osmosis Water (D).

### DETAILED DESCRIPTION

Disclosed herein is a composition and method for treating material and media contaminated with a pathogen such as SARS-CoV-2. Also disclosed herein is a composition and treatment regimen which can be used to address and treat patients presenting with symptoms associated with viral infections caused by coronaviruses including, but not limited to, SARS-CoV-2. Such viruses can be members of Coronaviridea including the subfamily Orthocoronavirinae (such as beta coronaviruses like SARS-CoV, SARS-CoV-2, MERS-CoV).

Coronavirus disease 2019 (COVID-19), due to infection with severe acute respiratory coronavirus 2 [SARS-CoV-2; also known as the 2019 novel coronavirus (2019-nCoV)], emerged in Wuhan, China in December 2019, and has resulted in a roughly 3% mortality rate. While this mortality rate is much higher than for influenza (roughly 0.05%), influenza-related hospitalizations and deaths in the US nevertheless are roughly 280,000 and 16,000, respectively, as a result of the much higher prevalence. COVID-19 has spread to all six major populated continents and is now a pandemic.

SARS-CoV-2 infection can lead to potentially lifelong or mortal consequences. The virus infects respiratory cells by targeting the receptor angiotensin converting enzyme (ACE). Onset of symptoms after exposure is approximately 4 days, with rapid progression often leading to hospitalization within 1-4 days thereafter.

Common symptoms include fever, myalgia, headache, diarrhea, shortness of breath, and cough with expectoration and possibly hemoptysis. Resting respiratory rate in more severely affected patients can exceed 30 breaths per minute, C reactive protein (CRP) levels can exceed 30 mg/L (normal levels are <3mg/L), and blood oxygen saturation falling below 93% [11]. Computed tomography (CT) scans can show rapidly developing subpleural ground-glass opacities (GGOs), with potential development of fibrosis. Poor prognosis is also associated with abnormal coagulation features.

At the outset of this pandemic there were no known approved treatments or preventions for COVID-19. And for SARS-CoV-2 virus infections. While several have been proposed and have become available in the ensuing months, additional effective post-infection treatments to minimize both short- and long-term clinical consequences are urgently needed

Also disclosed herein is a composition useful in addressing infection by one of more other microbiological pathogens. Non-limiting examples of such other microbiological pathogens include at least one of the following: pathogens such as those within the family Paramyxoviridae (such as measles morbillivirus), Herpesviridae (such as varicella-zoster virus); Mycobacteriaceae (such as mycobacterium tuberculosis); Orthomyxoviridae (such as influenzavirus A, influenzavirus B); Picornavivdae (such as enterovirus, poliovirus, coxsackie A viruses, coxsackie B viruses and the like); Calicivirdae (such as noroviruses); Adenoviridae and the like, Staphylococcaceae (such as staphyloccoccu aureus, like methicillin-resistant Staphylococcus aureus); Enterococcaceae (including vancomycin-resistant enterococci), Streptococcaceae (including streptococci) gram positive species such as Clostridioides difficile, Listeria, Coynehacterium and the like.

In the case of SARS-CoV-2, Coronaviruses constitute the subfamily Orthocoronavirinae, in the family Coronaviridae, order Nidopiales, and realm Riboviria. These are enveloped viruses with a positive-sense single-stranded RNA genome and a nucleocapsid of helical symmetry. The genome size of coronaviruses is one of the largest among RNA viruses. They have characteristic club-shaped spikes that project from their surface.

The present disclosure is based, in part on the unexpected discovery that vaporized or atomized material as disclosed herein having a pH less than 7, when introduced into contact with biological tissue, for example, respiratory tissue such as mammalian lung tissue, including but not limited to bronchial tissue, alveolar tissue, esophageal tissue, sinus tissue and the like in individuals presenting with such bacterial, viral, or fungal infection assocaited with respiratory tissue experience reduction in one or more symptoms including but not limited to congestion, tissue inflammation and the like. It has also been discovered that administration of vaporized or atomized material as disclosed herein can reduce microbial pathogen load assocaited that is assocaited with that tissue. Also disclosed is a method for treating infection in a subject that comprises the step of introducing an acidified vaporized composition into contact with lung tissue.

Also disclosed is a method for treating infection caused in whole or in part by one of the foregoing microbiological pathogens that can include the step of contacting at least one lung tissue region of a subject with a composition that includes a therapeutic material. In certain embodiments, the composition administered can present have a composition pH below 7. The composition may include a dilute acid selected from the group consisting of hydrochloric acid, nitric acid, phosphoric acid, chloric acid, perchloric acid, chromic acid, sulfuric acid, permanganic acid, prussic acid, bromic acid, hydrobromic acid, hydrofluoric acid, iodic acid, fluoboric acid, fluosilicic acid, fluotitanic acid and mixtures thereof. In certain embodiments, the composition can include sulfuric acid.

Where desired or required, the therapeutic material can also include between 100 and 1000 ppm of an inorganic ion selected from the group consisting of calcium, magnesium and mixtures thereof. In certain embodiments, the concentration of inorganic ion can be between 100 and 900 ppm; between 100 and 800 ppm; between 100 and 700 ppm; between 100 and 600 ppm; between 100 and 500 ppm; between 100 and 400 ppm; between 100 and 300 ppm; between 200 and 900 ppm; between 200 and 800 ppm; between 200 and 700 ppm; between 200 and 600 ppm; between 200 and 500 ppm; between 200 and 400 ppm; between 200 and 300 ppm; between 300 and 900 ppm; between 300 and 800 ppm; between 300 and 700 ppm; between 300 and 600 ppm; between 300 and 500 ppm; between 300 and 400 ppm.

In certain embodiments, the composition includes dissociated sulfuric acid molecules and at least one inorganic buffering ion such as calcium and has a pH as measured that is less than 7; less than 6; less than 5; less than 4; less than 3; less than 2; less than 1.

In certain embodiments, the therapeutic material that includes sulfuric acid and calcium ions can be produced by the process that comprises the steps of:

contacting a volume of a concentrated inorganic acid in liquid form having a molarity of at least 7, a density between 22° and 70° baume and a specific gravity between 1.18 and 1.93 in a reaction vessel with an inorganic hydroxide present in a volume sufficient to produce a solid material present in the resulting composition as at least one of a precipitate, a suspended solid, a colloidal suspension; and

removing the solid material from the resulting liquid material, wherein the resulting material is a viscous material having a molarity of 200 to 150 M.

The composition of matter as disclosed herein can be formed by the addition of a suitable inorganic hydroxide to a suitable inorganic acid. The inorganic acid may have a density between 22° and 70° baume; with specific gravities between about 1.18 and 1.93. In certain embodiments, it is contemplated that the inorganic acid will have a density between 50° and 67° baume; with specific gravities between 1.53 and 1.85. The inorganic acid can be either a monoatomic acid or a polyatomic acid.

The inorganic acid employed can be homogenous or can be a mixture of various acid compounds that fall within the defined parameters. It is also contemplated that the acid may be a mixture that includes one or more acid compounds that fall outside the contemplated parameters but in combination with other materials will provide an average acid composition value in the range specified. The inorganic acid or acids employed can be of any suitable grade or purity. In certain instances, tech grade and/or food grade material can be employed successfully in various applications. When required, the inorganic acid or acids can be of higher purity grade.

In preparing the product herein, the inorganic acid component can be contained in any suitable reaction vessel in liquid form at any suitable volume. In various embodiments, it is contemplated that the reaction vessel can be non-reactive beaker of suitable volume. The volume of acid employed can be as small as 50 ml. Larger volumes up to and including 5000 gallons or greater are also considered to be within the purview of this disclosure.

The inorganic acid can be maintained in the reaction vessel at a suitable temperature such as a temperature at or around ambient. It is within the purview of this disclosure to maintain the initial inorganic acid in a range between approximately 23° and about 70° C. However lower temperatures in the range of 15° and about 40° C. can also be employed.

The inorganic acid is agitated by suitable means to impart mechanical energy in a range between approximately 0.5 HP and 3 HP with agitation levels imparting mechanical energy between 1 and 2.5 HP being employed in certain applications of the process. Agitation can be imparted by a variety of suitable mechanical means including, but not limited to, DC servo drive, electric impeller, magnetic stirrer, chemical inductor and the like.

Agitation can commence at an interval immediately prior to hydroxide addition and can continue for an interval during at least a portion of the hydroxide introduction step.

In the process as disclosed herein, the acid material of choice may be a concentrated acid with an average molarity (M) of at least 7 or above. In certain procedures, the average molarity will be at least 10 or above; with an average molarity between 7 and 10 being useful in certain applications. The acid material of choice employed may exist as a pure liquid, a liquid slurry or as an aqueous solution of the dissolved acid in essentially concentrated form.

Suitable acid materials can be either aqueous or non-aqueous materials. Non-limiting examples of suitable acid materials can include one or more of the following: hydrochloric acid, nitric acid, phosphoric acid, chloric acid, perchloric acid, chromic acid, sulfuric acid, permanganic acid, prussic acid, bromic acid, hydrobromic acid, hydrofluoric acid, iodic acid, fluoboric acid, fluosilicic acid, fluotitanic acid.

In certain embodiments, the defined volume of a liquid concentrated strong acid employed can be sulfuric acid having a specific gravity between 55° and 67° baume. This material can be placed in the reaction vessel and mechanically agitated at a temperature between 16° and 70° C.

In certain specific applications of the method disclosed, a measured, defined quantity of suitable hydroxide material can be added to an agitating acid, such as concentrated sulfuric acid, that is present in the non-reactive vessel in a measured, defined amount. The amount of hydroxide that is added will be that sufficient to produce a solid material that is present in the composition as a precipitate and/or a suspended solid or colloidal suspension. The hydroxide material employed can be a water-soluble or partially water-soluble inorganic hydroxide. Partially water-soluble hydroxides employed in the process as disclosed herein will generally be those which exhibit miscibility with the acid material to which they are added. Non-limiting examples of suitable partially water-soluble inorganic hydroxides will be those that exhibit at least 50% miscibility in the associated acid. The inorganic hydroxide can be either anhydrous or hydrated.

Non-limiting examples of water-soluble inorganic hydroxides include water soluble alkali metal hydroxides, alkaline earth metal hydroxides and rare earth hydroxides; either alone or in combination with one another. Other hydroxides are also considered to be within the purview of this disclosure. “Water-solubility” as the term is defined in conjunction with the hydroxide material that will be employed is defined a material exhibiting dissolution characteristics of 75% or greater in water at standard temperature and pressure. The hydroxide that is utilized typically is a liquid material that can be introduced into the acid material. The hydroxide can be introduced as a true solution, a suspension or a super-saturated slurry. In certain embodiments, it is contemplated that the concentration of the inorganic hydroxide in aqueous solution can be dependent on the concentration of the associated acid to which it is introduced. Non-limiting examples of suitable concentrations for the hydroxide material are hydroxide concentrations greater than 5 to 50% of a 5 mole material.

Suitable hydroxide materials include, but are not limited to, lithium hydroxide, sodium hydroxide, potassium hydroxide, ammonium hydroxide, calcium hydroxide, strontium hydroxide, barium hydroxide, magnesium hydroxide, and/or silver hydroxide. Inorganic hydroxide solutions when employed may have concentration of inorganic hydroxide between 5 and 50% of a 5-mole material, with concentration between 5 and 20% being employed in certain applications. The inorganic hydroxide material, in certain processes, can be calcium hydroxide in a suitable aqueous solution such as is present as slaked lime.

In the process as disclosed, the inorganic hydroxide in liquid or fluid form is introduced into the agitating acid material in one or more metered volumes over a defined interval to provide a defined resonance time. The resonance time in the process as outlined is considered to be the time interval necessary to promote and provide the environment in which the hydronium ion material as disclosed herein develops. The resonance time interval as employed in the process as disclosed herein is typically between 12 and 120 hours with resonance time intervals between 24 and 72 hours and increments therein being utilized in certain applications.

In various applications of the process, the inorganic hydroxide is introduced into the acid at the upper surface of the agitating volume in a plurality of metered volumes. Typically, the total amount of inorganic hydroxide material will be introduced as a plurality of measured portions over the resonance time interval. Front-loaded metered addition being employed in many instances. “Front-loaded metered addition”, as the term is used herein, is taken to mean addition of the total hydroxide volume with a greater portion being added during the initial portion of the resonance time. An initial percentage of the desired resonance time -considered to be between the first 25% and 50% of the total resonance time.

It is to be understood that the proportion of each metered volume that is added can be equal or can vary based on such non-limiting factors as external process conditions, in situ process conditions, specific material characteristics, and the like. It is contemplated that the number of metered volumes can be between 3 and 12. The interval between additions of each metered volume can be between 5 and 60 minutes in certain applications of the process as disclosed. The actual addition interval can be between 60 minutes to five hours in certain applications.

In certain applications of the process, a 100 ml volume of 5% weight per volume of calcium hydroxide material is added to 50 ml of 66° baume concentrated sulfuric acid in 5 metered increments of 2 ml per minute, with or without admixture. Addition of the hydroxide material to the sulfuric acid produces a material having increasing liquid turbidity. Increasing liquid turbidity is indicative of calcium sulfate solids forming as precipitate. The produced calcium sulfate can be removed in a fashion that is coordinated with continued hydroxide addition in order to provide a coordinated concentration of suspended and dissolved solids.

Without being bound to any theory, it is believed that the addition of calcium hydroxide to sulfuric acid in the manner defined herein may result in the consumption of the initial hydrogen proton or protons associated with the sulfuric acid resulting in hydrogen proton oxygenation such that the proton in question is not off-gassed as would be generally expected upon hydroxide addition. Instead, the proton or protons are recombined with ionic water molecule components present in the liquid material.

Where desired or required, after the suitable resonance time as defined has passed, the resulting material is subjected to a non-bi-polar magnetic field at a value greater than 2000 gauss; with magnetic fields great than 2 million gauss being employed in certain applications. It is contemplated that a magnetic field between 10,000 and 2 million gauss can be employed in certain situations. The magnetic field can be produced by various suitable means. One non-limiting example of a suitable magnetic field generator is found in U.S. Pat. No. 7,122,269 to Wurzburger, the specification of which is incorporated by reference herein.

Solid material generated during the process and present as precipitate or suspended solids can be removed by any suitable means. Such removal means include, but need not be limited to, the following: gravimetric, forced filtration, centrifuge, reverse osmosis and the like.

Solid material generated during the process and present as precipitate or suspended solids can be removed by any suitable means. Such removal means include, but need not be limited to, the following: gravimetric, forced filtration, centrifuge, reverse osmosis and the like.

The material produced by the process as disclosed can be present as a shelf-stable viscous liquid that is believed to be stable for at least one year when stored at ambient temperature and between 50 to 75% relative humidity. The stable electrolyte composition of matter can be use neat in various end use applications. The stable electrolyte composition of matter can have a 1.87 to 1.78 molar material that contains 8 to 9% of the total moles of acid protons that are not charged balanced.

The material produced by the process disclosed herein has molarity of 200 to 150 M strength, and 187 to 178 M strength in certain instances, when measured titrimetrically though hydrogen coulometry and via FTIR spectral analysis. The material has a gravimetric range greater than 1.15; with ranges greater than 1.9 in in certain instances. The material, when analyzed, is shown to yield up to1300 volumetric times of orthohydrogen per cubic ml versus hydrogen contained in a mole of water.

In certain embodiments, this material can be introduced into water to produce the therapeutic material employed herein. It is contemplated that the use solution that is produced will contain between 0.5 volume % and 10 volume %. In certain embodiments, the therapeutic material will contain between 0.5 and 8 volume %; between 0.5 and 7 volume %; between 0.5 and 6 volume %; between 0.5 and 5 volume %; between 0.5 volume %; between 0.5 and 4 volume %; between 0.5 and 3 volume %; between 0.5 and 2 volume %; between 0.5 and 1 volume %; between 1 and 10 volume % 1 and 8 volume %; between 1 and 7 volume %; between 1 and 6 volume %; between 1 and 5 volume %; between 1 volume %; between 1 and 4 volume %; between 1 and 3 volume %; between 1 and 2 volume %; between 2 and 10 volume % 2 and 8 volume %; between 2 and 7 volume %; between 2 and 6 volume %; between 2 and 5 volume %; between 2 and 4 volume %; between 2 and 3 volume %; between 2 and 10 volume % 2 and 8 volume %; between 2 and 7 volume %; between 2 and 6 volume %; between 2 and 5 volume %; between 2 and 4 volume %; between 2 and 3 volume %.

The process as outlined heretofore can produce compounds having the following general formula:

$$⌊ {H}_{x} {O}_{\frac{( x - 1 )}{2}} ⌋ {Z}_{y}$$

x is an odd integer ≥3;
y is an integer between 1 and 20; and
Z is one of a monoatomic ion from Groups 14 through 17 having a charge between −1 and −3 or a poly atomic ion having a charge between −1 and −3.

In the components as disclosed herein monatomic constituents that can be employed as Z include Group 17 halides such as fluoride, chloride, iodide and bromide; Group 15 materials such as nitrides and phosphides and Group 16 materials such as oxides and sulfides. Polyatomic constituents include carbonate, hydrogen carbonate, chromate, cyanide, nitride, nitrate, permanganate, phosphate, sulfate, sulfite, chlorite, perchlorate, hydrobromite, bromite, bromate, iodide, hydrogen sulfate, hydrogen sulfite. It is contemplated that the composition of matter can be composed of a single one to the materials listed above or can be a combination of one or more of the compounds listed. In certain embodiments, Z is sulfate.

It is also contemplated that, in certain embodiments, x is an integer between 3 and 9, with x being an integer between 3 and 6 in some embodiments.

In certain embodiments, y is an integer between 1 and 10; while in other embodiments y is an integer between 1 and 5.

In certain embodiments, x is an odd integer between 3 and 12; y is an integer between 1 and 20; and Z is one of a group 14 through 17 monoatomic ion having a charge between −1 and −3 or a poly atomic ion having a charge between −1 and −3 as outlined above, some embodiments having x between 3 and 9 and y being an integer between 1 and 5.

Where present, the ion complex as disclosed herein is believed to be stable and may be capable of functioning as an oxygen donor in the presence of the environment created to generate the same. The material may have any suitable structure and solvation that is generally stable and capable of functioning as an oxygen donor. Particular embodiments of the resulting solution will include a concentration of the ion as depicted by the following formula:

$$[ {H}_{x} {O}_{\frac{( x - 1 )}{2}} ] +$$

wherein x is an odd integer ≥3.

It is contemplated that ionic version of the compound as disclosed herein exists in unique ion complexes that have greater than seven hydrogen atoms in each individual ion complex which are referred to in this disclosure as hydronium ion complexes. As usedherein, the term “hydroniumion complex” can be broadly defined as the cluster of molecules that surround the cation HₓOₓ₋₁+where x is an integer greater than or equal to 3. The hydronium ion complex may include at least four additional hydrogen molecules and a stoichiometric proportion of oxygen molecules complexed thereto as water molecules. Thus, the formulaic representation of non-limiting examples of the hydronium ion complexes that can be employed in the process herein can be depicted by the formula:

$$[ {H}_{x} {O}_{\frac{( x - 1 )}{2}} + {( {H}_{2} O )}_{y} ]$$

where x is an odd integer of 3 or greater; and

y is an integer from 1 to 20, with y being an integer between 3 and 9 in certain embodiments.

In various embodiments disclosed herein, it is contemplated that at least a portion of the hydronium ion complexes will exist as solvated structures of hydronium ions having the formula:

H₅+xO_{2y}+

wherein x is an integer between 1 and 4; and

y is an integer between 0 and 2.

In such structures, an

$$[ {H}_{x} {O}_{\frac{( x - 1 )}{2}} ] +$$

core is protonated by multiple H₂O molecules. It is contemplated that the hydronium complexes present in the composition of matter as disclosed herein can exist as Eigen complex cations, Zundel complex cations or mixtures of the two. The Eigen solvation structure can have the hydronium ion at the center of an H₉O₄+ structure with the hydronium complex being strongly bonded to three neighboring water molecules. The Zundel solvation complex can be an H₅O₂+ complex in which the proton is shared equally by two water molecules. The solvation complexes typically exist in equilibrium between Eigen solvation structure and Zundel solvation structure. Heretofore, the respective solvation structure complexes generally existed in an equilibrium state that favors the Zundel solvation structure.

The present disclosure is based, at least in part, on the unexpected discovery that stable materials can be produced in which hydronium ion exists in an equilibrium state that favors the Eigen complex. The present disclosure is also predicated on the unexpected discovery that increases in the concentration of the Eigen complex in a process stream can provide a class of novel enhanced oxygen-donor oxonium materials.

The process stream as disclosed herein can have an Eigen solvation state to Zundel solvation state ratio between 1.2 to 1 and 15 to 1 in certain embodiments; with ratios between 1.2 to 1 and 5 to 1 in other embodiments.

The novel enhanced oxygen-donor oxonium material as disclosed herein can be generally described as a thermodynamically stable aqueous acid solution that is buffered with an excess of proton ions. In certain embodiments, the excess of protons ions can be in an amount between 10% and 50% excess hydrogen ions as measured by free hydrogen content.

In certain embodiments, the composition of matter can have the following chemical structure:

$$[ {H}_{x} {O}_{\frac{( x - 1 )}{2}} + {( {H}_{2} O )}_{y} ] Z$$

wherein x is an odd integer between 3-11;

y is an integer between 1 and 10; and

Z is a polyatomic ion or monoatomic ion.

The polyatomic ion can be derived from an ion derived from an acid having the ability to donate on or more protons. The associated acid can be one that would have a pKₐ values ≥1.7 at 23° C. The ion employed can be one having a charge of +2 or greater. Non-limiting examples of such ions include sulfate, carbonate, phosphate, oxalate, chromate, dichromate, pyrophosphate and mixtures thereof. In certain embodiments, it is contemplated that the polyatomic ion can be derived from mixtures that include polyatomic ion mixtures that include ions derived from acids having pKₐ values ≤1.7.

In certain embodiments, the compound is composed of a stoichiometrically balanced chemical composition of at least one of the following: hydrogen (1+), triaqua-μ3-oxotri sulfate (1:1); hydrogen (1+), triaqua-μ3-oxotri carbonate (1:1), hydrogen (1+), triaqua-μ3-oxotri phosphate, (1:1); hydrogen (1+), triaqua-μ3-oxotri oxalate (1:1); hydrogen (1+), triaqua-μ3-oxotri chromate (1:1) hydrogen (1+), triaqua-μ3-oxotri dichromate (1:1), hydrogen (1+), triaqua-μ3-oxotri pyrophosphate (1:1), and mixtures thereof in admixture with water. In certain embodiments, the compound is hydrogen (1+), triaqua-μ3-oxotri sulfate (1:1).

Also disclosed herein is a method for addressing a microbiological pathogenic infection caused by a virus such as members of Coronaviridea including the subfamily Orthocoronavirinae (such as beta coronaviruses like SARS-CoV, SARS-CoV-2, MERS-CoV). The method comprises the step of introducing the composition as disclosed herein into contact with tissue in the respiratory system such as epithelial tissue for a contact interval. The contact interval is one that results in a reduction of the microbiological pathogen assocaited with the human epithelial tissue.

The therapeutic composition can be introduced the respiratory system of a subject in a manner where at least a portion of the therapeutic composition comes into contact with respiratory tissue proximate thereto. It is believed that introduction of at least a portion of the therapeutic composition as disclosed herein into contact with target tissue to results in a reduction of the viral load present in or on that tissue. Non-limiting examples of respiratory tissue including but not limited to epithelial tissue such as that found in the alveoli, bronchi, esophagus, sinuses, nasal cavity and the like.

Disclosed is a method for addressing an infection by a microbiological pathogen comprising the step of introducing the composition as disclosed herein into contact with epithelial tissue for a contact interval, wherein the contact results in a reduction of at least one pathogen associated with the epithelial tissue as measured by pathogen load.

Where desired, the therapeutic material can be present in and administered as a component in a liquid, gel, ointment or the like. It is also contemplated that can be nebulized, aerosolized, particulized to facilitate administration to the affected region as would occur in respiratory system infections.

The method as disclosed herein contemplates the introduction one or more doses of the therapeutic composition into the respiratory system of a subject. The therapeutic composition can be present in a suitable carrier liquid that can be rendered suitable for inhalation. Where desired or required the composition present in a suitable carrier liquid material can be administered as a vaporous material, nebulized material, aerosolized material, particulate material or the like in order to facilitate administration and uptake by inhalation. Administration can occur by more direct application, in certain embodiments, as by swabbing, spraying rinsing, immersion, and the like.

Where materials are aerosolized or nebulized, the material can be processed into droplets having a size suitable for inhalation uptake. Non-limiting examples of suitable droplet size include droplets having sizes between 0.1 and 20 μm; between 0.1 and 18 μm; between 0.1 and 17μm; between 0.1 and 16 μm; between 0.1 and 15 μm; between 0.1 and 14 μm; between 0.1 and 13 μm; between 0.1 and 12 μm; between 0.1 and 12 μm; between 0.1 and 11 μm; between 0.1 and 10 μm; between 0.1 and 9 μm; between 0.1 and 8 μm; between 0.1 and 7 μm; between 0.1 and 6 μm; between 0.1 and 5 μm; between 0.1 and 4 μm; between 0.1 and 3 μm; between 0.1 and 2 μm; between 0.1 and 1 μm; between 0.1 and 0.5 μm; 0.5 and 20 μm; between 0.5 and 18 μm; between 0.5 and 17 μm; between 0.5 and 16 μm; between 0.5 and 15 μm; between 0.5 and 14 μm; between 0.5 and 13 μm; between 0.5 and 12 μm; between 0.5 and 12 μm; between 0.5 and 11 μm; between 0.5 and 10 μm; between 0.5 and 9 μm; between 0.5 and 8 μm; between 0.5 and 7 μm; between 0.5 and 6 μm; between 0.5 and 5 μm; between 0.5 and 4 μm; between 0.5 and 3 μm; between 0.5 and 2 μm; between 0.5 and 1 μm; between 1 and 20 μm; between 1 and 18 μm; between 1 and 17 μm; between 1 and 16 μm; between 1 and 15 μm; between 1 and 14 μm; between 1 and 13 μm; between 1 and 12 μm; between 1 and 11 μm; between 1 and 10 μm; between 1 and 9 μm; between 1 and 8 μm; between 1 and 7 μm; between 1 and 6 μm; between 1 and 5 μm; between 1 and 4 μm; between 1 and 3 μm; between 1 and 2 μm; between 2 and 20 μm; between 2 and 18 μm; between 2 and 17 μm; between land 16 μm; between 2 and 15 μm; between 2 and 14 μm; between 2 and 13 μm; between 2 and 12 μm; between 2 and 11 μm; between 2 and 10 μm; between 2 and 9 μm; between 2 and 8 μm; between 2 and 7 μm; between 2 and 6 μm; between 2 and 5 μm; between 2 and 4 μm; between 2 and 3.

The pathogen infection that can be treated and pathogen load reduced or eliminated can include but is not limited to pathogens such as those within the family Paramyxoviridae (such as measles morbillivirus), Herpesviridae (such as varicella-zoster virus); Mycobacteriaceae (such as mycobacterium tuberculosis); Orthomyxoviridae (such as influenzavirus A, influenzavirus B); Picornavivdae (such as enterovirus, poliovirus, coxsackie A viruses, coxsackie B viruses and the like); Calicivirdae (such as noroviruses); Adenoviridae and the like, Staphylococcaceae (such as staphyloccoccu aureus, like methicillin-resistant Staphylococcus aureus); Enterococcaceae (including vancomycin-resistant enterococci), Streptococcaceae (including streptococci) gram positive species such as Ciostridioides difficile, Listeria, Coynebacterium and the like.

In the case of SARS-CoV-2 infections, SARS-CoV-2 infection can lead to potentially lifelong or mortal consequences. The virus infects respiratory cells by targeting the receptor angiotensin converting enzyme (ACE). Onset of symptoms after exposure is approximately 4 days, with rapid progression often leading to hospitalization within 1-4 days thereafter. Common symptoms include fever, myalgia, headache, diarrhea, shortness of breath, and cough with expectoration and possibly hemoptysis. Resting respiratory rate in more severely affected patients can exceed 30 breaths per minute, C reactive protein (CRP) levels can exceed 30 mg/L (normal levels are <3mg/L), and blood oxygen saturation falling below 93%. Computed tomography (CT) scans can show rapidly developing subpleural ground-glass opacities (GGOs), with potential development of fibrosis. Poor prognosis is also associated with abnormal coagulation features.

It has been found, quite unexpectedly that the composition as disclosed herein can be administered at a low pH and provide an effective treatment to diseases such as those caused by SARS-CoV-2 without triggering damage or toxicity in affected tissue, the local surrounding tissue, assocaited tissue or in the system at large. The composition as disclosed herein exhibits unique antiviral properties and is effective against viruses such as those that cause COVID-19.

### EXAMPLE I

In order to test the efficacy of the composition as disclosed herein, material is produced by is prepared by placing 50 ml portions of concentrated liquid sulfuric acid having a mass fraction H₂ SO₄ of 98%, an average molarity(M) above 7 and a specific gravity of 66° baume in non-reactive vessels and maintaining them at 25° C. with agitation by a magnetic stirrer to impart mechanical energy of 1 HP to the liquid.

Once agitation has commenced, a measured quantity of sodium hydroxide is added to the upper surface of each portion of the agitating acid material. The sodium hydroxide material employed is a 20% aqueous solution of 5M calcium hydroxide and is introduced in five metered volumes introduced at a rate of 2 ml per minute over an interval of five hours with to provide a resonance time of 24 hours. The introduction interval for each metered volume is 30 minutes.

Turbidity is produced with addition of calcium hydroxide to the sulfuric acid indicating formation of calcium sulfate solids. The solids are permitted to precipitate periodically during the process and the precipitate removed from contact with the reacting solution.

Upon completion of the 24-hour resonance time, the resulting material is exposed to a non-bi-polar magnetic field of 2400 gauss resulting in the production of observable precipitate and suspended solids for an interval of 2 hours. The resulting material is centrifuged and force filtered to isolate the precipitate and suspended solids.

The samples are collected for future use. Test samples are subjected to FFTIR spectra analysis and titrated with hydrogen coulometry. The sample material has a molarity ranging from 200 to 150 M strength and 187 to 178 strength. The material has a gravimetric range greater than 1.15; with ranges greater than 1.9 in in certain instances. The composition is stable and has a 1.87 to 1.78 molar material that contains 8 to 9% of the total moles of acid protons that are not charged balanced. FTIR analysis indicates that the material has the formula hydrogen (1+), triaqua-μ3-oxotri sulfate (1:1). The material is also found to include dilute sulfuric acid and 400 ppm calcium ions.

### EXAMPLE II

A second embodiment of the liquid material as disclosed herein is prepared by introducing 50 ml units of concentrated liquid sulfuric acid having a mass fraction H₂SO4 of 98%, an average molarity(M) above 7 and a specific gravity of 66° baume into a non-reactive vessel and maintaining each at 25° C. with agitation by a magnetic stirrer to impart mechanical energy of 1 HP to the each liquid unit.

Once agitation has commenced, a measured quantity of sodium hydroxide is added to the upper surface of the agitating acid material of each liquids unit. The sodium hydroxide material employed is a 20% aqueous solution of 5M calcium hydroxide and is introduced in five metered volumes introduced at a rate of 2 ml per minute over an interval of five hours with to provide a resonance time of 24 hours. The introduction interval for each metered volume is 30 minutes.

Turbidity is produced with addition of calcium hydroxide to the sulfuric acid indicating formation of calcium sulfate solids. The solids in each unit are permitted to precipitate periodically during the process and the precipitate is removed from contact with the reacting solution.

Upon completion of the 24-hour resonance time, the resulting material is centrifuged and force filtered to isolate the precipitate and suspended solids from the liquid material and respective resulting material units are collected for further use and analysis.

### EXAMPLE III

A 5 ml portion of the material produced according to the method outlined in Example I is admixed in a 5 ml portion of deionized and distilled water at standard temperature and pressure. The excess hydrogen ion concentration is measured as greater than 15% by volume and the pH of the material is determined to be 1.

### EXAMPLE IV

To further evaluate the materials prepared in Examples I and II, samples of the materials are diluted with deionized water to provide material that contains 1% by volume of the respective material in water. These samples are evaluated against a dilute sulfuric acid solution, a dilute sulfuric acid solution with to which calcium sulfate is added to yield 300 ppm and a dilute sulfuric with 400 ppm calcium sulfate and well as a reverse osmosis water control.

All samples are diluted in an acid matrix for analysis. The testing is completed using a Thermo iCAP 6300 Duo ICP-OES for calcium and sulfur content following EPA method 200.7.

Each test material is initially prepared by simple dilution in a 5% nitric acid matrix. The calibration standards are prepared in the same acid matrix to match the samples. However, this preparation leads to high recoveries for calcium which is believed to be a result of the sulfuric acid present in the samples but not present in the calibration standards. The calibration standards are re-prepared with a small amount of sulfuric acid in order to match the samples, and the analysis repeated in order to provide better QC recoveries that approach 100%.

In order to test for conductivity the samples are each diluted with de-ionized water for analysis. The testing is completed using a Mettler Toledo Seven Excellence Meter with a conductivity probe following EPA method 120.1. Predicted conductivity results are presented in Table I.

TABLE I
Summary of Conductivity Results

| Sample Name | Conductivity, mS/cm |
|---|---|
| Dilute sulfuric acid | 556 |
| Example I Sample | 551 |
| Example II Sample | 552 |
| Reverse Osmosis Water | 3.2 (μS/cm) |
| Dilute Sulfuric Acid w/300 ppm CaSO₄ | 562 |
| Dilute Sulfuric Acid w/400 ppm CaSO₄ | 558 |

In order to evaluate freezing point, the samples are analyzed using a TA Instruments Q100 DSC equipped with an RCS-90 cooling system following USP <891>. Predicted results are presented in Table II.

TABLE II
Summary of Freeze Point Results

| | Melting |
|---|---|
| Sample Name | Temperature, ° C. |
| Dilute sulfuric acid | −8.73 |
| Example I | −9.07 |
| Example II | −9.05 |
| Reverse Osmosis Water | 0.83 |
| Dilute Sulfuric Acid w/400 ppm CaSO₄ | −9.27 |

The density and specific gravity of the samples are determined at 20° C. using an Anton Paar digital density meter following EPA method 830.7300. predicted results are presented in Table III.

TABLE III
Summary of Density and Specific Gravity Results

| | Density | Specific |
|---|---|---|
| Sample Name | g/cm³ | Gravity |
| Dilute sulfuric acid | 1.0384 | 1.0403 |
| Example I | 1.0403 | 1.0422 |
| Reverse Osmosis Water | 0.9982 | 1.0000 |
| Dilute Sulfuric Acid w/400 ppm CaSO₄ | 1.0400 | 1.0418 |

The samples are also titrated for hydrogen ion content with acidity being determined following ASTM D1067—Test Method A to a pH of 8.6. The testing was completed using a Metrohm 826 Titrando equipped with a pH probe. Predicted results are presented in Table IV.

TABLE IV
Summary of Acidity (Titration) Results

| Sample Name | Acidity @ pH 8.6, meq/L |
|---|---|
| Dilute sulfuric acid | 1276.76 |
| Example I | 1307.28 |
| Example II | 1305.00 |
| Reverse Osmosis Water | 0.08 |
| Dilute Sulfuric Acid w/300 ppm CaSO₄ | 1295.68 |
| Dilute Sulfuric Acid w/400 ppm CaSO₄ | 1260.36 |

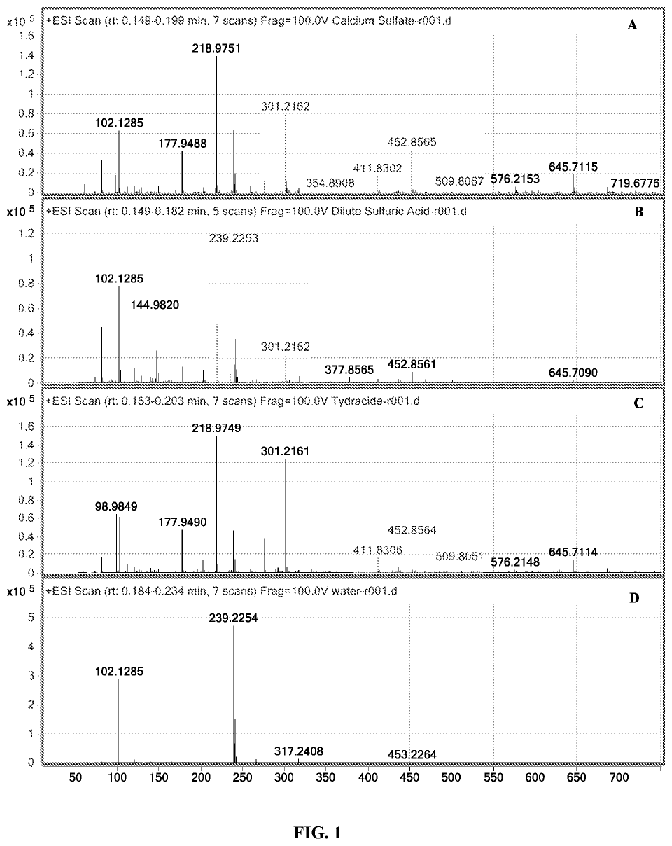

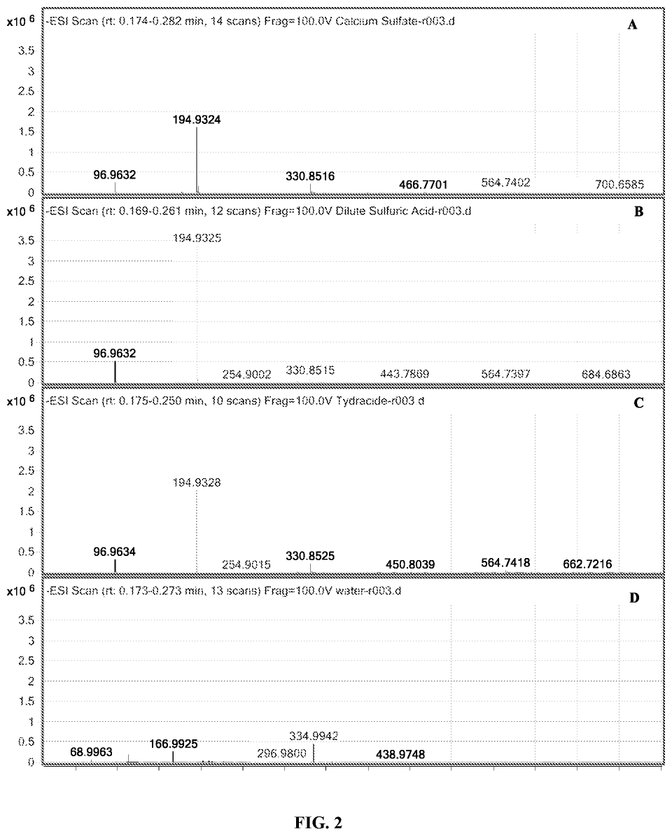

Solutions were analyzed an Agilent 1290/G6530 Q-TOF LC-MS using direct infusion (no column) and electrospray ionization in the positive and negative modes. Representative mass spectra collected in the positive and negative ionization modes are shown in FIGS. 1 and 2 with for Dilute Sulfuric Acid w/400 ppm CaSO₄ (A), Dilute Sulfuric Acid (B), Tydracide (C), and Reverse Osmosis Water (D).

### EXAMPLE V

The respective samples of Example I are diluted to produce 5 volume % of the product in water and are found to be shelf stable for at least 12 to 18 months. The excess hydrogen ion concentration is measured to be greater than 15% and the pH of the material is determined to be 1.

### EXAMPLE VI

Various studies have been conducted to explore the use of the compounds and compositions as disclosed herein. Material produced according the process outlined in Example II is determined to be able to function as a polar solvent, and when introduced into pure water, depending on its concentration, and can be adjusted to a final resulting stable water solution, with a pH as low as 0. Polar solvents containing the material produced exhibits the ability to destroy prokaryotic based bacteria, viruses, and fungi following brief periods of exposure, and have been also shown to be safe for eukaryotic based tissues that are known to maintain acid-base equilibrium.

### EXAMPLE VII

To ascertain performance of the material, solutions are prepared at final concentrations of material in water at of 2.5, 5, 15, 20 and 25 vol % respectively and added to SARS-CoV-2 containing media. The solution was allowed to incubate for the selected time periods of 1 minutes and 5 minutes. After incubation, a serial dilution was performed in Dulbecco's Modified Eagle's Medium (DMEM) and each dilution was screened using a viral plaque assay with VERO cells using Saline Solution as a control. The control Saline Solution showed no significant impact on SARS-CoV-2 survival, as measured as plaque forming units per ml (PFU/ml), after 1 minute and 5 minutes of exposure times. All samples of the material of Example II, from 2.5 vol % up to 25 vol % concentrations, showed effectiveness against the Coronavirus, SARS-CoV-2, after both 1 minute and 5 minutes of exposure; with over a 5 log reduction (>99.999%) in each instance in plaque forming units per ml (PFU/ml), with no detectable survival. The results are presented in Table V.

TABLE V
Stable Hydronium Test Results against SARS-CoV-2

| | 1:1 Stable Hydronium against SARS-CoV-2 | | | | | |
|---|---|---|---|---|---|---|
| | Control | Sample | Sample | Sample | Sample | Sample |
| | Sample | One | Two | Three | Four | Five |
| Concentra- | | 2.50% | 5.00% | 15.00% | 20.00% | 25.00% |
| tion | | | | | | |
| One Minute | 1.10E+05 | N/D | N/D | N/D | N/D | N/D |
| Log | | >5 log | >5 log | >5 log | >5 log | >5 log |
| Reduction | | | | | | |
| Five Minutes | 1.10E+05 | N/D | N/D | N/D | N/D | N/D |
| Log | | >5 log | >5 log | >5 log | >5 log | >5 log |
| Reduction | | | | | | |

N/D = non-detectable

### EXAMPLE VIII

Tests using the material outlined in Example VI was tested against the following viruses: herpes simplex virus type 1 (HSV-1) and type 2 (HSV-2) and feline calicivirus (employed as a norovirus surrogate) using standard cell culture or plaque assay techniques and was found to inactive the viruses listed.

### EXAMPLE IX

The Infectious Disease Society of America has identified a group of bacterial pathogens as the major cause of drug-resistant infections in healthcare facilities. The mnemonic “ESKAPE” was developed to easily identify the organisms that comprise this critical group, namely Enterococcus, Staphylococcus, Klebsiella, Acinetobacter, Pseudomonas, and ESBL (Enterobacter and E.coli). The solution as outlined in Example II was tested for its ability to neutralize the growth of ESKAPE pathogens in the laboratory setting. Using methods outlined by the Clinical Laboratory Standards Institute, three concentrations of the composition of Example II at 2.5 vol %, 1.0 vol % and 0.3 vol % respectively, were tested for bacterial growth inhibition. Growth inhibition of all multi-drug resistant ESKAPE pathogens tested, as well as clinical isolates from other bacteria general/species (including Haemophilus influenza, Stenotrophomonas maltophila, Citrobacter freundi, and Serratia marcescens) as part of a comprehensive activity survey of Gram positive and Gram negative organisms that cause clinically relevant infections, was achieved by either the 1 vol % or 2.5 vol % the Stable Hydronium solution.

### EXAMPLE X

Additional studies performed using the materials outlined in Example VII suggest that the action of the material as disclosed herein is not subject to common mechanisms of drug resistance. The laboratory experiments demonstrate the antibacterial activity of the Stable Hydronium against recalcitrant clinical isolates of ESKAPE pathogens and other disease-causing bacterial species of contemporary origin and phenotype.

### EXAMPLE XI

Tests were performed to determine the performance of the material as disclosed herein relative to sulfuric acid of similar pH given the low pH values the disclosed material can possess. The material as prepared according to the process outlined in Example II. Current US EPA guidelines (870.1000) require acute or short-term toxicity testing be performed on all registered chemicals according to their probable routes of human exposure. The toxicology tests (known as the standard “Six-Pack”) are performed on laboratory animals to simulate the human health impact of chemical substances. The tests for the solution containing the material disclosed in Example II were conducted according to EPA guidelines at a concentration of 50 vol %.

An Acute Dermal Toxicity Test was performed by applying a patch containing the test solution directly to the skin of healthy rats. According to the five-category rating Global Harmonized System for chemical classification (GHS), the Stable Hydronium solution is a Category 5 substance, i.e. practically non-toxic, and according to the four-category US EPA rating, is considered a Category 4 product, requiring no hazard statements to be present on the product label.

An Acute Oral Toxicity study was performed to determine the potential to produce toxicity from a single dose via oral administration to healthy rats. The data are consistent with assigning a GHS Oral Toxicity classification of Category 5, and an EPA classification of Category 4, essentially non-toxic.

An Acute Inhalation Toxicity study was performed where rats were continuously exposed to a test material aerosol (1-4 micron particle size) over a four-hour period. All animals survived exposure to the test atmosphere saturated with the solution and gained body weight during the study with no gross abnormalities seen during necropsy.

The test material solution was also evaluated in a Primary Skin Irritation study using the more sensitive rabbit species as a test subject. These data assign the test material solution to the lowest toxicity categories for skin irritation on both GHS and EPA scales.

Using a rabbit model, Primary Eye Irritation was measured by instillation of a 100 microliter drop of a 5% concentration of test material solution into one eye each of healthy animals, resulting in a total numerical score of 19.7 and classification of as moderately irritating to the eye (EPA Category 3, GHS Category 2B).

A Local Lymph Node Assay (LLNA) was performed in mice to determine if the test material solution had the capacity to sensitize rodent skin, directly measuring immune cell proliferation in the lymph nodes. The results showed that the test material solution is not considered to be a contact dermal sensitizer and would require no classification by GHS or EPA.

The results from these pivotal studies demonstrate that the solution containing the material as disclosed herein is effective against a number of viruses, as well as bacteria. The solution is considered safe according to the “Six-PC” EPA toxicity testing paradigm. Unlike traditional acid products that are highly corrosive and highly toxic, solution containing the material as disclosed herein can be assigned the lowest chemical toxicity rating in most categories, and is only moderately irritating to the eyes. Solutions containing the material as disclosed herein have the potential to be used safely and effectively as a technical grade ingredient for surface decontamination where low-cost, environmentally friendly, green chemistries are preferred. Due to the safety profile demonstrated on tissue surfaces, oral ingestion and lung inhalation, Solutions containing the material as disclosed herein also have the potential to be used therapeutically in patients with COVID-19.

### EXAMPLE XII

The 5 volume % material of Example II is diluted with distilled deionized water at a ratio of four parts water to 1part material and package in 2oz/60 ml glass bottles with droppers.

### EXAMPLE XIII

An aliquot of 2 ml each of the material as outlined in Example IV are introduced into a PARI nebulizer to produce a particle size of 2.5 μm that can be administered to each respective subject via inhalation though as suitable nebulizer mask. When the nebulizer is turned on, the material is suitable nebulized.

### EXAMPLE XIV

100 individuals with confirmed cases of COVID 19 as confirmed by PCR testing and presenting with various respiratory symptoms up to an including Acute Respiratory Distress each receive 2 ml doses, every 3 to 4 hours, 4 times daily (10 minute treatment intervals each) for 7 days via PARI nebulizer. To assess the efficacy of material as disclosed herein, subjects are randomized to either Arm A will receive the composition of Example IV (67 individuals) or Arm B while 33 condition and age matched subjects who receive placebo of normal saline solution. Treatment of each individual commences immediately upon confirmation of COVID 19 with follow up visits for 14 days post-treatment and at Weeks 3 and 4 after the completion of treatment and at Month 3 post-treatment.

The individuals treated with the composition of Examples II, XI, and XII are evaluated at Day 7 and at least 50% of the individuals demonstrate no respiratory symptoms. At Day 14, these individuals test negative for COVID-19 based on the standard PCR test.

### EXAMPLE XV

Animal studies are conducted to establish the safety of compositions composed of the composition prepared according the process of Example II in normal saline solution at concentration values of 0.1 vol %; 0.5 vol %; 0.75 vol %; 1.0 vol %; 2.0 vol % and 5.0 vol %, respectively.

In the study, test rats are employed and the respective compositions are delivered into the lungs of each test subject under conditions configured to simulate those produced by a PARI nebulizer in a human. Test subjects are dosed with the specific concentration for 10-minute intervals four times per day at three-hour intervals for up to 14 days. An additional group of test subjects is dosed with normal saline as a control under the same conditions. A control group is assembled receiving no inhalation dosage.

A portion of each test cohort is euthanized at 7 days, 10 days, and 14 days. The lung tissue is subjected to visual inspections as well as histological testing. No degradation of ling tissue is observed between the subjects receiving the test material and those receiving the saline treatments.

### EXAMPLE XV

An adult male presenting with a positive COVID test result as confirmed by PCR nasopharyngeal swab has an oxygen saturation value between 20 and 30%. The subject is treated with an inhaled composition of 0.5 vol % of the hydrogen (1+), triaqua-μ3-oxotri sulfate (1:1) composition prepared according to the process of Example I. The material is administered in 10 ml alequots over a 10-minute interval 4 times in a 24-hour interval. The oxygen saturation value at 24 hours is measured as 85%.

Treatment as outlined is continued for an additional 24 hours. PCR tests administered at 48 hours post treatment onset, 72 hours post treatment onset and 7 days post treatment onset and 14 days post treatment onset are negative for SARS-COV 2.

An adult female presenting with symptoms of ARDS is diagnosed as having COVID by PCR testing. The subject has an oxygen saturation of 50% as measured by pulse oximetry. The subject is treated with an inhaled composition of 0.75 vol % of the hydrogen (1+), triaqua-μ3-oxotri sulfate (1:1) composition prepared according to the process of Example I. The material is administered in 10 ml aliquots over a 10-minute interval 4 times in a 24-hour interval. The oxygen saturation value at 24 hours is measured as 90%.

Treatment as outlined is continued for an additional 24 hours. PCR tests administered at 48 hours post treatment onset, 72 hours post treatment onset and 7 days post treatment onset and 14 days post treatment onset are negative for SARS-COV 2.

### EXAMPLE XVI

An adult male presenting with symptoms of a respiratory infection and fever is diagnosed as having COVID by PCR testing. The subject has an oxygen saturation of 70% as measured by pulse oximetry. The subject is treated with an inhaled composition of 1. vol % of the hydrogen (1+), triaqua-μ3-oxotri sulfate (1:1) composition prepared according to the process of Example I. The material is administered in 10 ml aliquots over a 10-minute interval 4 times in a 24-hour interval. The oxygen saturation value at 24 hours is measured as 90%. The subject is afebrile at 36 hours post treatment onset.

Treatment as outlined is continued for an additional 24 hours. PCR tests administered at 48 hours post treatment onset, 72 hours post treatment onset and 7 days post treatment onset and 14 days post treatment onset are negative for SARS-COV 2.

### EXAMPLE XVII

A randomized controlled study is conducted to ascertain the efficacy of the composition as disclosed herein is conducted by enrolling 100 adult subjects acutely infected with SARS-CoV 2. Sixty-seven (67) subjects are treated according with the composition according to the method as disclosed herein administered by inhalation. Thirty-three condition and age matched subjects are treated with normal saline administered by inhalation. Follow up visits are conducted daily for 14 days and at Week 3 and 4 after treatment and at Month 3 for each subject.

Enrollment inclusion criteria include the following factors: Male or female or any race or ethnicity; at least 50 years of age; confirmed diagnosis of infection with coronavirus. Exclusion criteria include various pre-existing health conditions including respiratory or metabolic acidosis.

In addition to appropriate safety end points, each subject is assessed for one or more of the following end points; time to RT-PCR test negativity as well as time to return to normal Oxygen Saturation (5PO2) levels; Pneumonia severity index, time to restore normal temperature from fever; time to clinical improvement after infection; and various serum biomarkers such as C-reactive peptide (CRP); D-dimer; troponin; ferritin; surfactant protein D; angiopoietin-2 (Ang-2); macrophage migration inhibitory factor (MIF); extracellular nicotinamide phosphoribosyltransferase (eNAMPT); sphingosine 1-phosphate receptor 3 (S1PR3), cytokines including interleukin-1β(IL-1β, interleukin-6 (IL-6), interleukin-8 (IL-8), and tumor necrosis factor-α(TNF-α); interleukin-1 receptor antagonist (IL-lra).

Each enrolled subject is randomized to Arm 1 or Arm 2. Those randomized to Arm 1 will receive four times daily 2 cc of 0.5 vol % the hydrogen (1+), triaqua-μ3-oxotri sulfate (1:1) composition prepared according to the process of Example I in Normal saline every 3 to 4 hours (10-minute treatment each) via nebulizer for a 7-day period. Those randomized to Arm 2 will receive Normal Saline will be delivered three times daily, 2 cc every 3-4 hours (10 minutes treatments each via nebulizer for a 7-day period. Administration will be by PARI nebulizer.

The subjects enrolled in Arm 1 receiving the composition as disclosed herein experience abatement of symptoms, usually within 7 days of onset of treatment including negative PCR tests.

### EXAMPLE XVIII

The procedure outlined in Example XVII are repeated for concentrations of the composition as disclosed herein at 0.75% by volume and 1.0% by volume with similar results.

### EXAMPLE XIX

In order to support that the proposed composition is efficacious against microbiological pathogens other than virus SARS-Cov-2, Minimum inhibitory concentration (MIC) evaluation is performed against S. aureus ATCC 6538, S. aureus ATCC 33951, P. aeruginosa ATCC,1544, and P. aeruginosa ATCC BAA 2018 were performed using the material as prepared in Example I. the results are presented in Table VI and VII a supporting the MIC efficacy of the material as disclosed herein.

TABLE VI
Serial dilution of 10% solution with distilled water

| | Serial dilution % | | | | | | | | | |
|---|---|---|---|---|---|---|---|---|---|---|
| | 1 | 2 | 3 | 4 | 5 | 6 | 7 | 8 | 9 | 10 |
| 10% | 1.00% | 0.90% | 0.81% | 0.73% | 0.66% | 0.59% | 0.53% | 0.48% | 0.43% | 0.39% |
| pH | 1.8 | 2.1 | 2.4 | 2.8 | 3.1 | 3.5 | 3.7 | 3.9 | 4.1 | 4.3 |

TABLE VII
MIC results

| | challenge microbe | | | | | | | | | |
|---|---|---|---|---|---|---|---|---|---|---|
| | 1 | 2 | 3 | 4 | 5 | 6 | 7 | 8 | 9 | 10 |
| S. aureus | 1.815 | 2.068 | 2.401 | 2.759 | 3.126 | 3.455 | 3.72 | 3.9 | 4.12 | 4.26 |
| ATTC6538 | | | | | | | | | | |
| S. aureus | 1.815 | 2.068 | 2.401 | 2.759 | 3.126 | 3.455 | 3.72 | 3.9 | 4.07 | 4.26 |
| ATTC33951 | | | | | | | | | | |
| (antibiotic | | | | | | | | | | |
| resistant) | | | | | | | | | | |
| P. aeruginosa | 1.84 | 2.056 | 2.363 | 2.716 | 3.081 | 3.417 | 3.667 | 3.9 | 4.07 | 4.30 |
| ATCC 1544 | | | | | | | | | | |
| P. aeruginosa | 1.815 | 2.296 | 2.672 | 3.011 | 3.402 | 3.66 | 3.89 | 4.11 | 4.32 | 4.469 |
| ATCC BAA | | | | | | | | | | |
| 2018 | | | | | | | | | | |
| (antibiotic | | | | | | | | | | |
| resistant) | | | | | | | | | | |

While the invention has been described in connection with what is presently considered to be the most practical and preferred embodiment, it is to be understood that the invention is not to be limited to the disclosed embodiments but, on the contrary, is intended to cover various modifications and equivalent arrangements included within the spirit and scope of the appended claims, which scope is to be accorded the broadest interpretation so as to encompass all such modifications and equivalent structures as is permitted under the law.

What is claimed is:

1. A therapeutic material comprising:
a product produced by the process comprising the steps of:
contacting a volume of a concentrated inorganic acid in liquid form having a molarity of at least 7, a density between 22° and 70° baume and a specific gravity between 1.18 and 1.93 in a reaction vessel with an inorganic hydroxide present in a volume sufficient to produce a solid material present in the resulting composition as at least one of a precipitate, a suspended solid, a colloidal suspension; and
removing the solid material from the resulting liquid material, wherein the resulting material is a viscous material having a molarity of 200 to 150 M; and
water, wherein the therapeutic material has a pH less than 7.

2. The therapeutic material of claim 1 wherein the pH is less than 5.

3. The therapeutic material of claim 1 wherein the material further contains a dilute acid selected from the group consisting of hydrochloric acid, nitric acid, phosphoric acid, chloric acid, perchloric acid, chromic acid, sulfuric acid, permanganic acid, prussic acid, bromic acid, hydrobromic acid, hydrofluoric acid, iodic acid, fluoboric acid, fluosilicic acid, fluotitanic acid and mixtures thereof.

4. The therapeutic material of claim 2 wherein the dilute acid is sulfuric acid.

5. The therapeutic material of claim 1 where the product by process further comprises between 100 and 1000 ppm of an inorganic ion selected from the group consisting of calcium, magnesium and mixtures thereof.

6. The therapeutic material of claim 1 wherein the product is present in water in a concentration between 0.25% by volume and 5% by volume.

7. The therapeutic material of claim 5 wherein the product is present in water in a concentration between 0.5% by volume and 2% by volume.

8. The therapeutic material of claim 1 wherein the product is compound having the general formula:
$$[ {H}_{x} {O}_{\frac{( x - 1 )}{2}} + {( {H}_{2} O )}_{y} ] Z$$
wherein x is an odd integer >3;
y is an integer between 1 and 20; and
Z is a polyatomic ion or monoatomic ion.

9. The therapeutic material of claim 7 wherein Z is one of a monoatomic ion from Groups 14 through 17 having a charge value between −1 and −3 or a polyatomic ion having a charge between −1 and −3 and x is an integer between 3 and 11 and y is an integer between 1 and 10.

10. The therapeutic material of claim 8 wherein Z is selected from the group consisting of sulfate, carbonate, phosphate, oxalate, chromate, dichromate, pyrophosphate and mixtures thereof.

11. The therapeutic material of claim 1 composed of a stoichiometrically balanced chemical composition of at least one of the following: hydrogen (1+), triaqua-μ3-oxotri sulfate (1:1); hydrogen (1+), triaqua-μ3-oxotri carbonate (1:1), hydrogen (1+), triaqua-μ3-oxotri phosphate, (1:1); hydrogen (1+), triaqua-μ3-oxotri oxalate (1:1); hydrogen (1+), triaqua-μ3-oxotri chromate (1:1) hydrogen (1+), triaqua-μ3-oxotri dichromate (1:1), hydrogen (1+), triaqua-μ3-oxotri pyrophosphate (1:1), and mixtures thereof.

12. The therapeutic material of claim 1 wherein the therapeutic material is active against one or more microbiological pathogens present in a human body.

13. The therapeutic material of claim 12 wherein the one or more microbiological pathogens are present in the one or more locations in the respiratory system of a mammal.

14. A method for addressing a microbiological pathogenic infection in a patient, wherein the microbiological pathogenic infection is caused by at least one microbiological pathogen, the method comprising the step of;
introducing the composition of claim 1 into contact with epithelial tissue for a contact interval, wherein the contact results in a reduction of at least one microbiological pathogen assocaited with the human epithelial tissue.

15. The method of claim 14 wherein the composition is introduced as a liquid in at least one dose.

16. The method of claim 14 wherein the composition is introduced in at least one dose administration into contact with the epithelial tissue as droplets, the droplets having an average droplet size between 0.1 and 20 μm.

17. The method of claim 14 wherein the epithelial tissue is present in the respiratory system of a mammal.

18. The method of claim 17 wherein the epithelial tissue is present in at least one of the sinus cavities, bronchus, alveoli of a patient.

19. The method of claim 14 wherein the microbiological pathogen is at least one of the following: pathogens such as those within the family Paramyxoviridae (such as measles morbillivirus), Herpesviridae (such as varicella-zoster virus); Mycobacteriaceae (such as mycobacterium tuberculosis); Orthomyxoviridae (such as influenzavirus A, influenzavirus B); Picornavivdae (such as enterovirus, poliovirus, coxsackie A viruses, coxsackie B viruses and the like); Calicivirdae (such as noroviruses); Coronaviridea including the subfamily Orthocoronavirinae (such as beta coronaviruses like SARS-CoV, SARS-CoV-2, MERS-CoV); Adenoviridae and the like, Staphylococcaceae (such as staphyloccoccu aureus, like methicillin-resistant Staphylococcus aureus); Enterococcaceae (including vancomycin-resistant enterococci), Streptococcaceae (including streptococci) gram positive species such as Clostridioides difficile, Listeria, Coynebacterium and the like.

20. The method of claim 14 wherein the microbiological pathogen is SARS-CoV-2.
